(19) United States
(12) Patent Application Publication
(10) Pub. No.: US 2025/0331838 A1
(43) Pub. Date: Oct. 30, 2025

(54) DEPLOYABLE RETRACTOR

(71) Applicant: Morpheus AG, Spaichingen (DE)

(72) Inventors: Carsten LINTI, Stuttgart (DE); Simon BURMEISTER, Stuttgart (DE); Benjamin POHL, Esslingen (DE); Christoph RIETHMÜLLER, Leonberg (DE); Andreas SCHERRIEBLE, Ludwigsburg (DE); Cornelia FANO, Kirchheim (DE); Timo RACK, Rietheim-Weilheim (DE)

(73) Assignee: Morpheus AG, Spaichingen (DE)

(21) Appl. No.: 18/855,386

(22) PCT Filed: May 16, 2023
(86) PCT No.: PCT/EP2023/063161

§ 371 Date: Oct. 9, 2024

(30) Foreign Application Priority Data
May 18, 2022 (DE) 10 2022 112 521.1

(51) Int. Cl. A61B 17/02; A61B 17/00

(52) U.S. Cl. CPC A61B 17/0293; A61B 2017/00539; A61B 2017/00544; A61B 2017/00557

(57) ABSTRACT

A deployable retractor (1) with a folded-in configuration and a folded-out configuration is provided. In the folded-out configuration, the refractor (1) forms a completely encased channel (2), and the casing (3) of the channel (2) includes an at least partly double-walled textile (4) that surrounds a fluid interior (6) which has at least one fluid chamber (5) and can be filled with a fluid via a connection (7) and which is emptied in the folded-in configuration and is filled with the fluid in the folded-out configuration.

### CROSS-REFERENCE TO RELATED APPLICATIONS

This application is a 371 National phase of PCT/EP2023/063161, filed May 16, 2023, which claims priority from German Patent Application No. 10 2022 112 521.1, filed May 18, 2022, both of which are incorporated herein by reference as if fully set forth.

### TECHNICAL FIELD

The invention relates to a retractor, a method for producing a retractor and a method for operating a retractor.

### BACKGROUND

Retractors are used in operations such as spinal surgery. In order to perform an operation on the spine, the surgeon needs a channel to have a view of the operating field and to bring surgical instruments into the operating field.

To do this, the skin and the underlying tissue are usually cut open and widened. A retractor is then used to keep the skin and tissue apart. A retractor forms a channel on the inside through which the surgeon can then insert the instruments and also has a direct view of the surgical field.

To hold the tissue apart, two metallic, rigid, opposing spreader blades are usually inserted into the incision area and pushed apart using a metallic rod system until the skin opening is the desired size. It is also known to introduce a second set of spreader blades that are arranged orthogonally to the first set.

Since such retractors only hold the tissue apart in a few places, they create very strong pressure points, which can kill tissue during longer operations, some of which can last several hours.

### SUMMARY

Against this background, the invention is based on the object of creating a retractor that is gentle on tissue.

To solve the problem, one or more of the features disclosed herein are provided according to the invention. In particular, a deployable retractor is thus provided according to the invention for solving this object, which has a folded-in configuration and a folded-out configuration. In the folded-out configuration, the retractor forms a completely encased channel, wherein the casing of the channel comprises a textile. The textile is at least partially double-walled. The textile encloses a fluid interior which has at least one fluid chamber. The fluid interior can be filled with a fluid via a connection. Furthermore, the fluid interior is emptied in the folded-in configuration and filled with the fluid in the folded-out configuration.

When unfilled, the textile is folded in and only takes up a small volume, so that the textile can be easily inserted into the tissue opened for the operation. When the textile is then filled with the fluid, it expands and unfolds to form the channel. In this way, the surrounding tissue is evenly loaded and thus protected. Even during long operations, the tissue held apart is therefore sufficiently supplied with blood and is not killed. The technical solution according to the invention therefore creates a particularly gentle retractor.

The double-walled textile can be characterized in that the textile has an inner wall and an outer wall, wherein the fluid interior is formed between the outer wall and the inner wall. The textile may also have one or more intermediate walls between the inner and outer walls. Such a multilayer textile is also referred to as double-walled in the context of this description of the invention.

The textile can, but does not have to be completely double-walled. It is sufficient if it is at least partially double-walled. In addition to the areas in which the outer and inner walls are spaced apart in the folded-out configuration, there may also be contact areas in which the outer and inner walls touch, are sewn together and/or are interwoven even in the folded-out configuration. The contact areas can define the edges of fluid chambers, as described in more detail below.

A fluid can be a liquid, preferably water, or a gas, preferably air.

The folded-out configuration is one in which the fluid interior is filled with the fluid under a working pressure and thus forms the channel. The folded-in configuration is one in which the fluid interior is emptied and the pressure in the fluid interior corresponds to the external pressure.

The working pressure is preferably more than one or more bar and can particularly preferably be up to 15 bar or exactly 15 bar.

When the connection is closed, the fluid interior is preferably hermetically sealed and, in particular, watertight and/or airtight to the outside.

The fluid interior has a connection and thus exactly one or more than one connection. The exact one or more connections is or are preferably closable. Preferably, the fluid interior has exactly two connections. Two connections can facilitate the filling and emptying of the fluid interior, in particular if the fluid is a liquid, since air inclusions can then be avoided or removed in this way.

Even if the textile should have more than one fluid interior, more than one connection will be useful, preferably exactly one and particularly preferably exactly two connections per fluid interior.

A retractor designed according to the invention can be used for spinal operations, for example, but also for other surgical procedures.

Preferably, the fluid is a liquid. The particular advantage of using a liquid instead of a gas is that liquids are incompressible. Such hydraulic textiles offer the advantage over pneumatic textiles that the retractor can be formed to be dimensionally stable in the folded-out configuration. Surprisingly, it has been shown that such retractors can achieve the same hardness as conventional plastic tubes.

In an advantageous design of the retractor, it may be provided that the channel is dimensionally stable in the folded-out configuration. In particular, it may be provided that the textile has a predetermined, stable unfolding shape, which is reached at a working pressure, for example the working pressure already mentioned, and which is not further expanded by a further increase in the fluid pressure.

Preferably, the textile, in particular the fabric, has maximum expansion.

It may be provided that the textile is inelastic and/or inextensible at least in the folded-out configuration. Here, inelastic and unstretchable are not to be understood in an absolute sense, but in such a way that in the folded-out configuration, in which the fluid is under a working pressure, for example the aforementioned working pressure, an external application of pressure to the textile does not cause any deformation of the textile when the retractor is used as intended, so that the textile is dimensionally stable in the folded-out configuration. This therefore relates to external pressures that can arise when human tissue is held apart by the retractor. The same applies to the previously mentioned feature of dimensional stability.

The textile can also be inelastic and/or unstretchable in the folded-in configuration. Unlike in the folded-out state, the textile is not dimensionally stable, as the fluid interior is not filled and therefore no displacement of liquid is caused when the shape changes.

However, it is also possible that the textile is still elastic and/or stretchable in the folded-in state. To achieve a stable shape in the filled state, it is sufficient if the elasticity and/or stretchability decreases considerably at a pressure that is below or equal to the working pressure. This can be the case, for example, if a weaving technique is used in which the woven fabric can be stretched without the application of force, but which can no longer be stretched further above a certain degree of stretch.

The material, i.e. in particular the yarn from which the textile is made, is preferably an inelastic yarn. However, it can also have elastic properties, which can be influenced by the weaving technique used.

The preferred textile material is a high-strength synthetic fiber such as an aramid, particularly preferably a p-aramid fiber or a p-aramid copolymer fiber.

It may be provided that the outer and inner walls of the double-walled textile are deflected or sewn in the area opposite the connection.

In a further advantageous design of the retractor, it may be provided that the fluid interior has a plurality of fluid chambers. Preferably, the fluid chambers are in fluid-dynamic, in particular hydrodynamic, exchange with each other. A fluid can therefore flow from one fluid chamber into a neighboring fluid chamber. Surprisingly, it has been shown that such a multi-chamber system can lead to a particularly stable folded-out configuration and that at the same time the tissue held apart is particularly protected due to the uneven surface that is formed.

Preferably, the fluid exchange between adjacent fluid chambers is effected by lines formed in and/or by the textile.

The fluid exchange between neighboring fluid chambers can be bidirectional or unidirectional. A unidirectional exchange can be achieved, for example, via a membrane that is only permeable in one direction.

In a further advantageous design of the retractor, it may be provided that the textile has contact areas in which an outer and an inner wall of the textile are joined together. The connection can be made, for example, by seams and/or by weaving. Preferably, the contact areas are formed on a surface of the casing. The contact areas can form separation areas for the fluid chambers.

In order to enable a secure fit in the skin tissue without the retractor slipping out, it may be provided in a further advantageous design of the retractor that the channel is waisted in the folded-out configuration. Waisted can be characterized by the fact that the retractor is narrower between the channel ends. In this case, the channel can have a concave curvature rather than a cylindrical shape. The casing can particularly preferably have the shape of a hyperboloid.

Depending on the application, the channel in the folded-out configuration can alternatively be frustoconical, tapering downwards or upwards, or have a different shape.

It is preferably provided that the mold described in more detail below has a surface that is adapted to the desired design of the channel.

In a further advantageous design of the retractor, it may be provided that the casing has a support plate in a partial area. It has been found that a support plate leads to a particularly controlled unfolding. In use, the support plate can be brought into contact with a bone, for example, so that the retractor can be supported on the bone. During spinal surgery, for example, the support plate can be supported on the spinous process of a vertebra.

If there is no support in the body on a bone, the retractor can either be inserted unsupported or it can be held in place by a holding arm, wherein the holding arm can be attached to the support plate.

The support plate is preferably made of plastic, but can also be made of another material such as metal.

The support plate is preferably rigid. This means that the support plate remains dimensionally stable under the forces applied during intended use.

The textile is preferably attached to the support plate. It can be attached by gluing, for example, which can be particularly advantageous if the support plate only consists of a single plate.

The support plate is preferably designed as a double plate, wherein the textile is inserted, in particular clamped, between the two partial plates of the double plate. The partial plates can preferably have means for generating a contact pressure for this purpose. The two partial plates can be screwed together using screws, for example.

In order to improve the impermeability of the textile, a further advantageous design of the retractor may provide for the textile to be coated with a liquid-impermeable coating. The coating is preferably made of a plastic and/or an elastomer.

Preferably, the coating is applied to the inner walls of the fluid interior. Alternatively or additionally, the coating is applied to the outside of the textile.

The coating is preferably air-permeable. This ensures that any air pockets that may form in the fluid interior can be removed and that the textile remains airtight at the same time.

It may be provided that the casing is uneven on the outside in the folded-out configuration. Preferably, the casing has elevations and/or indentations on the outside. The feature that the surface of the textile is not smooth but has a surface structure can be realized, for example, by the retractor being designed as a multi-chamber system as described above, the fluid chambers of which inflate during filling, while the contact areas of the textile are not filled with the fluid. This gives the textile an uneven structure, which has the advantage that the fabric is less stressed overall and is better supplied with blood. Preferably, the uneven structure is caused by elevations that are arranged in a regular pattern.

In order to enable optimum illumination of the operating area, it may be provided that illuminants are arranged on or in an inner side of the casing. The illuminants can be designed as LEDs. Alternatively, light can be introduced via plastic or glass fibers. The illuminants can also be arranged, in particular mounted, on the inside of the support plate in the channel. The illuminants can also be arranged in or on the textile, in particular as ring lighting, in particular attached or woven in.

Means can be provided to ensure that the pressure of the fluid in the textile is increased in a controlled manner and to prevent a maximum pressure from being exceeded.

For example, a sensor can be provided with which a fluid pressure is measured.

It may be provided that the textile has a plurality of chambers, only some of which are filled with the fluid in the folded-out configuration.

Furthermore, it may be provided that a spring is formed which absorbs the counterpressure generated by the fabric pressing on the retractor and which prevents additional fluid, which can be injected by means of a syringe for example, from entering the textile if a predetermined reference pressure value is exceeded.

It may be provided that the retractor tapers downwards in the folded and/or unfolded state. Downwards is the direction in which the retractor is inserted into the fabric. This can be on a side of the channel facing away from the connection or connections.

To solve this object, one or more of the features disclosed herein directed to a method for producing a deployable retractor are also provided in accordance with the invention. Thus, in particular, in order to solve this object in a method for producing a deployable retractor which is designed according to the invention, in particular as described above and/or according to one of the protective claims directed to a retractor, it is proposed that the double-walled textile having the fluid interior is produced from a textile material and that the fluid interior is then coated. The textile can, for example, be produced by processing a textile fabric. For example, a textile fabric can be sewn and/or woven so that the double-walled textile with the fluid interior is formed. Preferably, the textile is coated on the outside with an airtight film before the fluid interior is coated. It may be provided that the airtight film is removed again after the coating has been applied. Such a method can be particularly advantageous if a retractor is manufactured that has a plurality of fluid chambers.

A flat textile structure can be designed, for example, as a fabric, woven fabric, knitted fabric, braided fabric, sewn fabric, non-woven fabric and/or felt.

For example, it may be provided that a multi-chamber textile fabric is produced in a first step. This fabric can then be surrounded on the outside with an airtight film in a second step. This film can be used to prevent a gas from escaping from the textile, which is then introduced into the fluid interior in a subsequent step. The introduction of a gas under sufficient gas pressure causes the fluid chambers to inflate. Once the chambers are inflated, a curable material in the liquid state, for example a silicone, an elastomer, in particular a vulcanizable elastomer, or a chemically curing material, including a two-component material, can be introduced into the inflated fluid chambers. This material then settles on the inner walls of the fluid chambers and then begins to harden. The fact that the fluid chambers are inflated during this time prevents the fluid chambers from sticking together. To ensure that the material reaches everywhere, the retractor is preferably rotated in different directions. This ensures that the material is applied evenly on the inside.

To solve the object, one or more of the features disclosed herein directed to a method for producing a deployable retractor are also provided in accordance with the invention. Thus, in particular, in order to solve this object in a method for producing a deployable retractor which is formed according to the invention, in particular as described above and/or according to one of the claims directed to a retractor, it is proposed that a flat textile structure, such as in particular a textile fabric, is coated and that the double-walled textile having the fluid interior is subsequently formed.

In a preferred design of the method described above for producing a deployable retractor, it may be provided that a material of the coating is meltable, that two layers of the textile structure are placed on top of each other so that their coated sides touch, that the meltable material is melted by heating at least in one area and that the two layers are pressed together at least in a partial area of the melted area.

The two layers can be produced, for example, by coating a layer of textile with the material and then cutting the coated textile into suitable pieces. Two such parts can then be placed on top of each other as described above. Alternatively, a cut textile part can also be folded so that the two layers are placed on top of each other. It is also conceivable that the final shape of the textile is formed by turning the textile inside out so that the laminate, which is initially applied to the outside, is then formed on the inside of the fluid interior. Such a method can preferably be used with a textile that only has a single fluid chamber. It is not necessary to cut the textile if the textile parts are already provided in the desired size. Cutting can optionally also take place at a later stage. For example, the double-walled textile can be produced for several retractors at the same time and then cut into the size required for the individual retractor.

The material of the coating is preferably a thermoplastic. It can be a thermoplastic polyurethane, for example.

In a further development of the method for producing a deployable retractor, it may be provided that a mold with a surface structure is provided, which has raised surfaces between which recesses are formed, and that the mold is pressed onto the two layers of the textile structure laid on top of one another. The mold is preferably heated before or during the pressing of the mold onto the two layers. The mold is preferably made of metal, such as brass. Furthermore, the mold is preferably designed as a plate. A surface of the mold or plate is preferably flat, but may also have a concavity or other curvature.

If the already heated mold is pressed onto the two layers as described, the coating material melts on the raised surfaces of the mold. The pressure exerted causes the molten material to bond together on the raised surfaces. After cooling, the two layers are then bonded to each other in these partial areas. For cooling, it is not necessary for the mold to continue to be pressed onto the textile, but it is also not impossible for cooling to take place while contact pressure is still being applied.

The surface structure of the mold therefore determines the areas in which the two layers are joined together. The connection only takes place in the areas of the raised surfaces. The two layers are not connected to each other in the recessed areas. The recesses therefore correspond to the chambers and the fluidic connections between the chambers and the raised surfaces form boundaries for the chambers. The method therefore allows the production of textile double-walled casings with a wide variety of chamber geometries.

The raised surfaces preferably have narrow webs. This maximizes the area in which the chambers can be filled with fluid.

After the two layers have been joined together at the intended points, the textile can be cut into its final shape.

It may be provided that the mold has a plurality of preferably repeating surface patterns. In this case, textile can be produced in a pressing process, which is then used to manufacture more than one retractor by cutting the textile into the required individual parts.

In an advantageous design of the aforementioned methods or independently thereof, a method for producing a deployable retractor may provide for contact areas to be formed to create a plurality of fluid chambers by interweaving and/or sewing together an outer and an inner wall of the textile. This is preferably carried out before the textile is coated.

To produce the multiple fluid chambers, the textile can be woven, knitted or sewn. Sewing is preferably carried out before the textile is coated. This prevents the coating from being perforated and leaking during the sewing process.

To solve the object, one or more of the features disclosed herein directed to a method for operating a deployable retractor are also provided in accordance with the invention. Thus, in particular, in order to solve this object in a method for operating a deployable retractor which is designed according to the invention, in particular as described above and/or according to one of the claims directed to a retractor, it is proposed that a syringe which has a fluid reservoir is connected to the connection and that the fluid interior is filled with the fluid by actuating the syringe. Preferably, the fluid is pressurized with a working pressure. The syringe is preferably designed as a mechanical manual syringe. The syringe is preferably connected to the connection via a hose. Such a method can be carried out particularly cost-effectively.

The liquid supply unit, such as the syringe or a liquid pump, can be connected to the connection(s) via a hose or hoses.

The connections can be connected to the liquid supply unit via medical plastic connections, for example.

The connection or connections can each be closed by a valve. For example, this can be a hose valve, a 2-way or 3-way stopcock. A Luer lock connection can also be used for the connection.

In an advantageous design of a method for operating a deployable retractor as described above or also independently of the design of the method described above, it may be provided that a sensor is connected to the connection, with which a fluid pressure is measured and/or that a control device is connected to the connection, with which a working pressure of the fluid, for example the working pressure already mentioned above, is set and controlled.

### BRIEF DESCRIPTION OF THE DRAWINGS

The invention will now be described in more detail with reference to one or a few exemplary embodiments, but is not limited to these few exemplary embodiments. Further exemplary embodiments and variants of the invention are obtained by combining the features of individual or multiple claims with one another and/or with individual or multiple features of the exemplary embodiments or the variants of the invention described above, wherein:

FIG. 1A to FIG. 1C show an exemplary embodiment of a deployable retractor according to the invention in folded-out configuration from different viewing directions,

FIG. 2A to FIG. 2C show the retractor shown in FIG. 1A to 1C in folded-in configuration from different viewing directions,

FIG. 3A to FIG. 3C show a further exemplary embodiment of a deployable retractor formed according to the invention in a folded-out configuration from different viewing directions,

FIG. 4A to FIG. 4D show the retractor shown in FIG. 3A to 3C in folded-in configuration from different viewing directions.

### DETAILED DESCRIPTION

In the following description, elements with the same function are given the same reference signs even if they have a different design or shape.

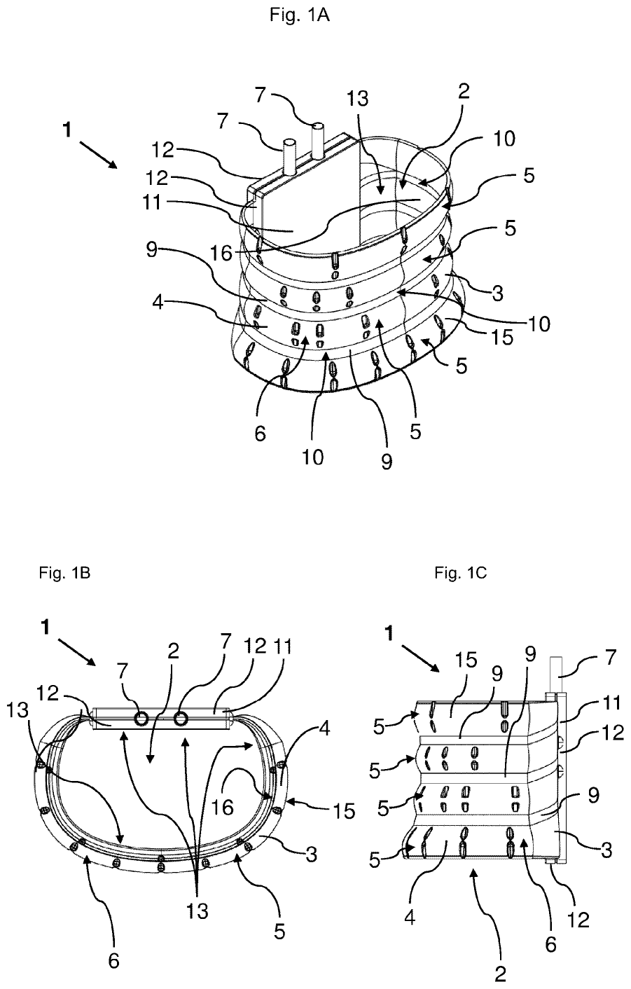

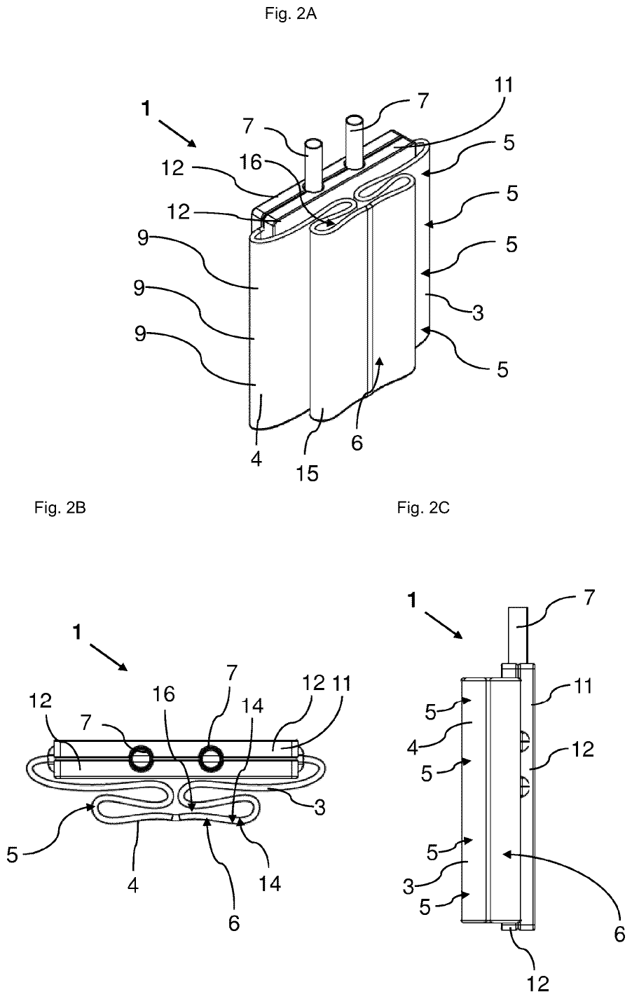

FIGS. 1A-1C and FIGS. 2A-2C show a deployable retractor 1. The retractor 1 has a support plate 11, which is designed as a double plate with two partial plates 12. A rectangular textile 4, which is made of a fabric woven from a p-aramid fiber, is clamped between the partial plates 12 with a first edge on a first side of the support plate 11 and clamped with a second edge parallel to the first edge on a side of the support plate 11 opposite the first side of the support plate 11. The two partial plates 12 are screwed together to achieve the clamping effect.

In the folded-out configuration, this forms a channel 2 that is completely encased. The casing 3 is formed by the support plate 11 and the textile 4.

The textile 4 is double-layered. It has an outer wall 15 and an inner wall 16. The outer wall 15 and the inner wall 16 enclose a fluid interior 6, so that the textile 4 can be filled with a fluid. In the exemplary embodiment shown, the retractor 1 is designed to be filled with a liquid, in particular water.

The textile 4 has two connections 7, which open into the fluid interior 6. The fluid interior 6 has a number of fluid chambers 5 that communicate with each other in terms of flow.

The fluid chambers 5 are separated from each other by contact areas 9. In the exemplary embodiment shown, the fluid chambers 5 and the contact areas 9 are formed circumferentially around the channel 2. In the exemplary embodiment described, the contact areas 9 are created by the outer wall 15 and the inner wall 16 being joined together with a seam in these areas. However, the seam is not completely circumferential around the casing 3. At one or more points, the outer wall 15 is not sewn to the inner wall 16, so that connecting lines 10 are formed, which connect adjacent fluid chambers 5 to each other.

The fluid chambers 5 are surrounded by the inner sides of the outer wall 15 and inner wall 16 as well as the boundaries created by the contact area 9. This border is coated on the inside with a hardened plastic coating 14, wherein the rotational coating process described above can be used for coating 14. The coating 14 is impermeable to the fluid, so that the fluid interior 6 is hermetically sealed when the connections 7 are closed.

The connections 7 can have a valve function to close them. For example, one of the connections 7 can be connected to a syringe or another liquid supply unit by means of a Luer lock connection in order to supply the liquid.

If the fluid interior 6 is not filled with fluid and thus emptied, the retractor 1 is in the folded configuration shown in FIGS. 2A to 2C. In this configuration, the geometry of the retractor 1 follows the geometry of the support plate 11. Since this is flat, the retractor in the folded configuration can be gently inserted into a skin incision and brought to the surgical field in the patient's body.

A syringe filled with the fluid can then be connected to one of the connections 7 directly and/or via a hose, depending on its design. The syringe can then be used to introduce fluid into the fluid chamber 6. This fills the fluid chambers 5 with the fluid. The surgeon performing the operation manually operates the hand syringe. The force applied by hand creates a working pressure of the fluid in the fluid chamber 6. The tube is then removed from the connection 7 and the connections 7 are both closed. As the fluid interior 6 is hermetically sealed, the fluid pressure is maintained.

Filling the fluid chambers 5 with the fluid causes the retractor 1 to unfold and take on the waisted shape shown in FIG. 1A to FIG. 1C. In the folded-out configuration, the retractor 1 is dimensionally stable under the working pressure. The dimensional stability is achieved by using an incompressible fluid on the one hand and by the fact that the textile fabric forming the outer wall 15 and the inner wall 16 is inelastic and non-stretchable under the working pressure compared to a counterpressure applied by the human tissue held apart during the operation.

In order to provide the surgeon with a good view of the surgical area in the patient's body, LEDs are attached to the inside of the channel 2 as illuminants 13 on the inside of the partial plate 12 and/or woven into the inner wall 16.

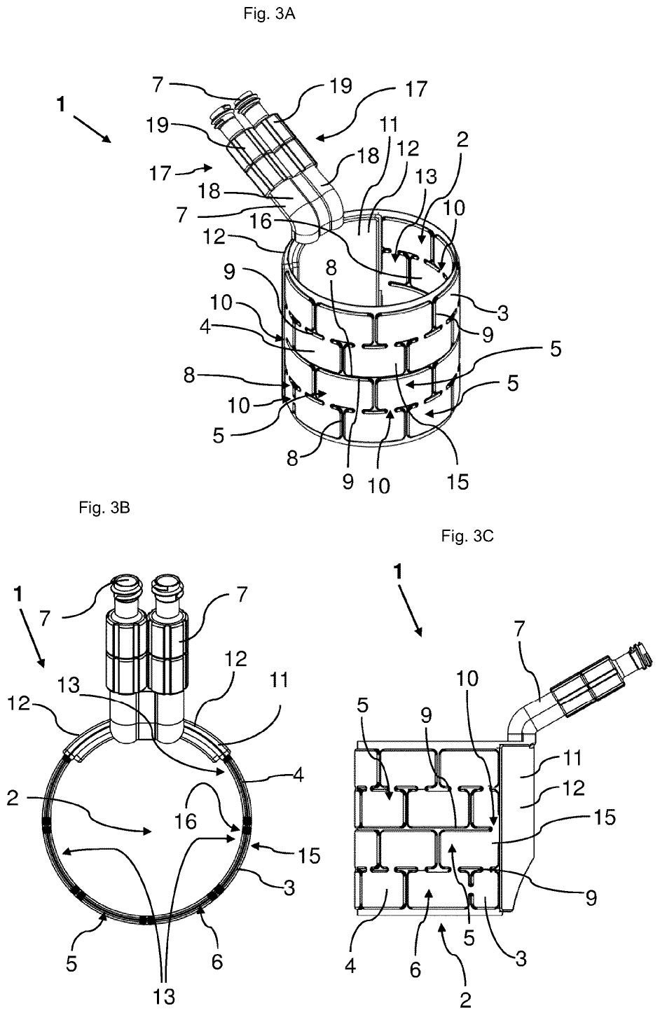

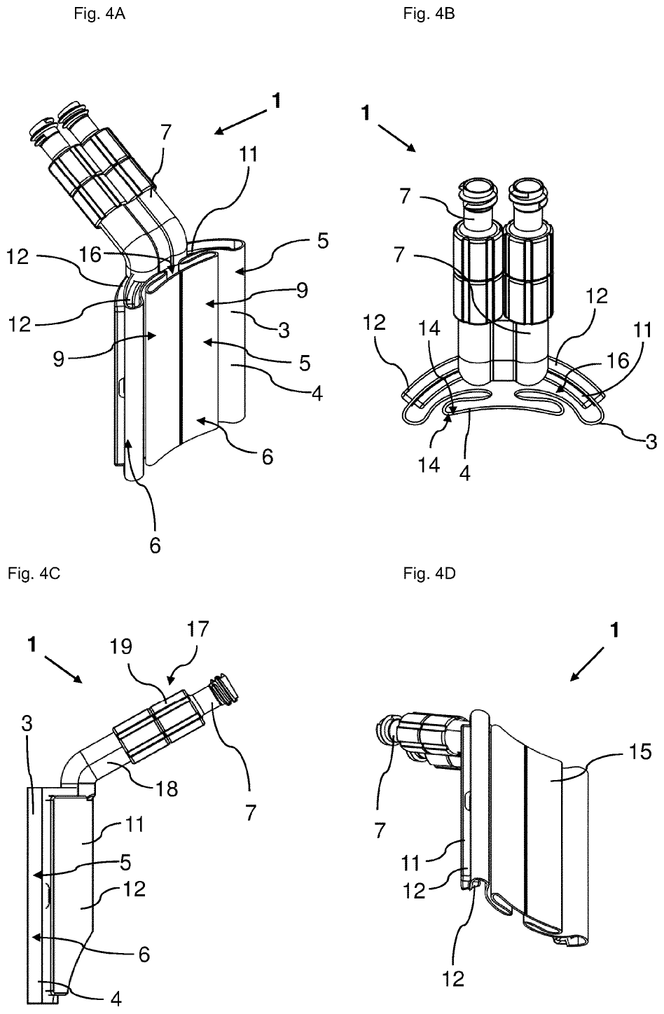

FIGS. 3A-3C and FIGS. 4A-4D show a second exemplary embodiment of a retractor 1 designed according to the invention. In the following, only the differences to the retractor 1 shown in FIGS. 1A-1C and FIGS. 2A-2C and described above will be discussed. Apart from these differences, the two exemplary embodiments are of the same design, so that, apart from the following remarks, the above remarks on the retractor 1 shown in FIGS. 1A-1C and FIGS. 2A-2C also apply accordingly to the retractor 1 shown in FIGS. 3A-3C and FIGS. 4A-4D. The retractor 1 shown in FIGS. 3A-3C and FIGS. 4A-4D has a support plate 11 that is curved. In the exemplary embodiment shown, the support plate 11 is curved in the shape of an arc. This also applies accordingly to the two partial plates 12. In the folded-out state shown in FIGS. 3A-3C, the channel 2 is cylindrical. It is not waisted; in an alternative exemplary embodiment, however, the channel 2 can also be waisted. In the folded-in state, the retractor 1 tapers in the lower area. This is the area that is the first to be inserted into the tissue when the retractor 1 is inserted. In the exemplary embodiment shown here, this is opposite the connections 7. The taper makes it easier to insert the retractor 1 into the fabric.

The connections 7 are angled. They have a valve 17 with which the connection lines 18 can be closed and opened. In the exemplary embodiment shown, this can be done manually. For this purpose, the valve 17 has a twist lock 19.

The retractor 1 has fluid chambers 5. It has a plurality of fluid chambers 5. In alternative exemplary embodiments, the retractor 1 can also have fewer fluid chambers 5. The fluid chambers 5 are each bordered by seams 8. The seams 8 cause the outer wall 15 and inner wall 16 to touch, thus forming contact areas 9. The seam 8 surrounding a fluid chamber 5 is interrupted at at least one point and thus forms a connecting line 10 through which the fluid can enter and exit the fluid chamber 5, so that the fluid chamber 5 is filled or emptied.

In summary, the invention relates to a deployable retractor 1 having a folded-in configuration and a folded-out configuration, wherein the retractor 1 in the folded-out configuration forms a completely encased channel 2, wherein the casing 3 of the channel 2 comprises an at least partially double-walled textile 4 which encloses a fluid interior 6 having at least one fluid chamber 5, which can be filled with a fluid via a connection 7 and which is emptied in the folded-in configuration and filled with the fluid in the folded-out configuration.

### LIST OF REFERENCE SIGNS

1 Retractor
2 Channel
3 Casing
4 Textile
5 Fluid chamber
6 Fluid interior
7 Connection
8 Seam
9 Contact area
10 Connecting line
11 Support plate
12 Partial plate
13 Illuminants
14 Coating
15 Outer wall
16 Inner wall
17 Valve
18 Connection lines
19 Twist lock

1. A deployable retractor (1), comprising: a casing (3) having a folded-in configuration and a folded-out configuration, in the folded-out configuration the casing (3) forms a completely encased channel (2); and
the casing (3) comprises an at least partially double-walled textile (4), which encloses a fluid interior (6) having at least one fluid chamber (5), which is fillable with a fluid via a connection (7) and which is emptied in the folded-in configuration and filled with the fluid in the folded-out configuration.

2. The deployable retractor (1) according to claim 1, wherein the fluid is a liquid.

3. The deployable retractor (1) according to claim 1. wherein at least one of a) the channel (2) is dimensionally stable in the folded-out configuration, or b) the textile (4) is at least one of inelastic or inextensible at least in the folded-out configuration.

4. The deployable retractor (1) according to claim 1, wherein the fluid interior (6) has a plurality of fluid chambers (5).

5. The deployable retractor (1) according to claim 1. wherein the textile (4) has contact areas (9) in which an outer and an inner wall of the textile (4) are connected to one another.

6. The deployable retractor (1) according to claim 1. wherein the channel (2) is waisted in the folded-out configuration.

7. The deployable retractor (1) according to claim 1. wherein the casing (3) has a support plate (11) in a partial area, in particular wherein the support plate (11) is designed as a double plate (11).

8. The deployable retractor (1) according to claim 1, wherein the textile (4) is coated with a liquid-impermeable coating (14).

9. The deployable retractor (1) according to claim 1. wherein the casing (3) in the folded-out configuration is uneven on an outside thereof and includes at least one of elevations or indentations.

10. The deployable retractor (1) according to claim 1. further comprising illuminants (13) arranged on or in an inner side of the casing (3)

11. A method for producing a deployable retractor (1), the method comprising:
providing a casing (3) having a folded-in configuration and a folded-out configuration, in the folded-out configuration the casing (3) forms a completely encased channel (2), and the casing (3) comprising a double-walled textile (4) that encloses a fluid interior (6) having at least one fluid chamber (5), and producing the fluid interior (6) from a textile material; and
coating the fluid interior (6) wherein the textile (4) is coated on the outside with an airtight film before the fluid interior (6) is coated.

12. The method for producing the deployable retractor (1) according to claim 11, wherein a flat textile structure is coated and the method further comprises subsequently forming the double-walled textile (4) having the fluid interior (6) from the flat textile structure that is coated.

13. The method for producing the deployable retractor (1) according to claim 12, wherein a material of the coating is meltable, two layers of the textile structure are placed on top of one another such that coated sides thereof touch, the meltable material is melted by heating at least in one area, and the two layers are pressed together at least in a partial area of the melted area.

14. The method for producing the deployable retractor (1) according to claim 13, further comprising providing a mold with a surface structure is provided which has raised surfaces between which recesses are formed, and pressing the mold onto the two layers of the textile structure laid on top of one another.

15. The method for producing the deployable retractor (1) according to claim 11, further comprising forming contact areas (9) to form a plurality of the fluid chambers (5) by at least one of interweaving or sewing together an outer and an inner wall of the textile (4).

16. A method for operating the deployable retractor (1) according to claim 1, the method comprising connecting a syringe with a fluid reservoir to the connection (7), and filling the fluid interior (6) with the fluid by actuating the syringe.

17. The method for operating the deployable retractor (1) according to claim 16, wherein a sensor is connected to the connection (7), and the method further comprises at least one of measuring a fluid pressure or connecting a control device to the connection (7), with which a working pressure of the fluid is set and controlled.

18. The deployable retractor (1) according to claim 4, wherein the fluid chambers (5) are in fluid-dynamic exchange with one another.

19. The deployable retractor (1) according to claim 7, wherein the support plate (11) comprises a double plate (11), and the textile (4) is inserted between the two partial plates (12) of the double plate (11).

20. The deployable retractor (1) according to claim 8, wherein the coating (14) is applied to at least one of inner walls of the fluid interior (6) or an outside of the textile (4).
